(19) United States
(12) Patent Application Publication
(10) Pub. No.: US 2002/0173688 A1
(43) Pub. Date: Nov. 21, 2002

(54) Ultrasonic treatment device and methods thereof

(76) Inventors: Peter Cheng Chen, Irving, CA (US); Toshimasa Tokuno, Tokyo, JP (US); Weng-Kwen Raymond Chia, Irvine, CA (US); Cary Hata, Tustin, CA (US)

Correspondence Address: Weng-Kwen Raymond Chia, 2382 Morse, Irvine, CA, 92614, US

(21) Appl. No.: 09/859,157

(22) Filed: May 15, 2001

(51) Int. Cl. A61N 5/02

(52) U.S. Cl. 600/2

(57) ABSTRACT

This invention discloses a method for treating tissue of a varicose vein, the method comprising applying ultrasonic vibrational energy targeting said varicose vein effective for stimulating and restoring tissue physiological functions of said vein. A catheter for treating tissue of a blood vessel comprises at least one ultrasound transducer element and at least a suction element configured and adapted for pulling the tissue toward the catheter radially, wherein the ultrasonic vibrational energy is provided by an ultrasound transducer element connected to an adjustable high frequency current source.

### FIELD OF THE INVENTION

The present invention generally relates to the improved system for ultrasonically treating tissue, more particularly, this invention relates to a catheter system and methods for applying ultrasonic vibrational energy targeting the venous blood vessel effective for stimulating and restoring tissue physiological functions of the blood vessel.

### BACKGROUND OF THE INVENTION

The human blood circulation is a closed system characterized by high pressure in the arteries and low pressure in the veins. Capillaries separate one type of pressure from the other. Pressure, especially in the veins, also is influenced by gravity and by the contraction of venous muscle. In the standing position hydrostatic pressure is added to resting pressure. Abnormally high pressure and tissue deterioration are primary reasons for vessel dilatation.

Since the pumping action of the heart is much more like that of a pressure pump than a suction pump, other mechanisms are needed to obtain an adequate venous return and thus a reduction of venous pressure. The mechanisms for venous return may include (1) vis a tergo (force from behind), (2) vis a fronte (force from in front), (3) venous tonus, and (4) muscle pump. The muscle pump is the most important mechanism for venous return to the heart. With insufficient valve function or reduced muscle activity, the degree of pressure reduction will not occur.

Varicose veins are dilated, elongated and tortuous veins in the subcutaneous tissue. The valves are incompetent so that blood flow is not predominantly in one direction as in normal veins but may be temporarily centrifugal, depending upon posture, venous pressure and such factors as muscular activity. Such reflux may occur when position or rate of flow is altered. Varicosities are most common in the lower extremities but they also occur in other anatomical areas. Some 10-20% of the world population may have varicose veins in the legs.

Valvular incompetence between the deep and superficial systems is the most important observation in primary varicosities. This factor appears to be the most important in determining the clinical course and progression of varicose veins. The fundamental abnormality is progressive, sequential incompetence of the valvular rings in the main superficial trunks and in the communicating veins. More proximal incompetent valves result in high venous pressure being applied to the more distal valves so that, over time, segmental dilatation of that venous segment and of the next distal valvular ring takes place.

The weak wall hypothesis suggests that a developmental weakness of the vein wall results in progressive venous dilatation even at normal venous pressures with secondary failure of competence of the dilated valvular rings. Secondary varicosities develop after either damage, obstruction, or both in the deep venous system. Damage occurs when, in the wake of a deep venous thrombosis, recanalization of the thrombosed veins may be associated with destruction of the valve cusps so that pressure in the involved segment is increased and under certain conditions flow may be temporarily reversed. The vein stretches and incompetence may develop in the valves which connect the deep and superficial systems. Obstruction of a major proximal segment such as the inferior vena cava, iliac or femoral veins may produce an important increase in venous pressure. The deep system distal to the obstruction is then exposed to an increased pressure which has two effects: dilatation of collateral channels and deep to superficial valvular incompetence, with increased pressure in the superficial system.

Both in valvular damage and in obstruction the end result is that the superficial system dilates to give rise to secondary varicosities. Their formation is aided by the fact that these veins have only weak external support from the subcutaneous tissue in that they are superficial to the deep fascia of the leg.

The accuracy of the clinical examination of varicose veins is limited and subjective. Three different modalities of treatment are commonly used: non-operative management, such as drugs or physical measures; surgery; and sclerotherapy. However, none of the above methods provides means for stimulating the tissue of the veins with ultrasonic vibrational energy by a minimally invasive treatment approach. Of particular interest to the present invention are ultrasonic energy therapeutic protocols, which have been proven to be highly effective in tissue ablation, while exposing a patient to minimal side effects and risks.

The traditional radiofrequency ablation provides therapeutic energy by heat conduction at a limited contact point while the ultrasonic vibrational energy can provide a deeper energy penetration over a target region by remote energy transmissions. By stimulating the tissues deeply and broadly, and causing the muscle to restore its tightness, the varicosity can be slowly reversed and cured.

Marcus et al. in U.S. Pat No. 5,295,484 and Castellano et al. in U.S. Pat No. 5,606,974 teach a catheter system having ultrasonic devices for intracardiac ablation of arrhythmias. However, neither patent discloses a medical device having ultrasonic vibrational energy therapy in venous treatment, particularly in varicose treatment.

Diederich et al. in U.S. Pat. No. 6,117,101 discloses an ultrasound ablation element secured to the distal section at a fixed position within an expandable member, wherein the ultrasound ablation element is adapted to emit a substantially circumferential pattern of ultrasound energy and to ablatively couple to the substantial portion of the circumferential region of tissue engaged by the expandable member in the radially expanded condition when the ultrasound ablation element is coupled to and actuated by an ultrasound ablation actuator. Diederich et al. does not disclose ultrasonic vibrational energy for stimulating venous tissue and restoring its physiological functions, such as muscular pumping activities.

Lesh in U.S. Pat. No. 6,164,283 discloses a method for treating atrial arrhythmia in a patient, comprising forming a circumferential conduction block in a circumferential region of tissue of a pulmonary vein, wherein the circumferential conduction block is formed without contacting the tissue with an ablative fluid medium by ultrasound ablation energy. However, Lesh does not disclose ultrasonic vibrational energy for stimulating tissue muscle and restoring its physiological functions.

There is therefore a clinical need for a device which comprises ultrasonic vibrational energy for stimulating tissue of a venous blood vessel and restoring its physiological functions to treat varicosity or other indications.

### SUMMARY OF THE INVENTION

In general, it is an object of the present invention to provide a method and a catheter system for stimulating a tissue or tissue muscle using ultrasonic vibrational energy. It is another object of the present invention to provide a method for treating tissue of a varicose vein, the method comprising applying ultrasonic vibrational energy targeting said varicose vein effective for stimulating and restoring tissue physiological functions of said vein.

Another object of the present invention is to provide a catheter for treating tissue of a blood vessel comprising at least one ultrasound transducer element and at least a suction element adapted for pulling the tissue toward the catheter radially, wherein the ultrasonic vibrational energy is effective to treat the tissue.

In still another embodiment, the object of the present invention is to provide a method for therapeutically treating a tissue, the method comprising applying ultrasonic vibrational energy toward the tissue effective for transmitting said vibrational energy onto said tissue, wherein the vibrational energy is controlled by an adjustable and controllable high frequency generator.

It is a further object of the present invention to provide a method for dispersing the ultrasonic vibrational energy through a fluid medium of an expandable chamber, wherein the expandable chamber encloses the ultrasound transducer element.

In one ab interno embodiment, the present invention comprises at least one ultrasonic transducer element mounted on a distal end portion of a device. The ultrasonic transducer element may be a single crystal transducer or a phased array crystal transducer. Ultrasonic transducer element configured and adapted for use in the invention is that capable of generating frequencies in the 1-100 MHz range under an applied electrical energy of 1 watt or above. The ultrasonic transducer element is typically composed of relatively brittle piezoelectric crystalline material that is somewhat fragile. The ultrasonic transducer element may be manufactured in different shape and size. In one embodiment, the ultrasound transducer element further comprises ultrasound crystals adapted to generate at least one of focused ultrasound energy or diffused ultrasound energy. The adjustable high frequency generator is capable of adjusting the current/frequency output manually or by a pre-programmed algorithm.

In another ab externo embodiment, the ultrasonic vibrational energy is applied from around the target blood vessel, wherein the ultrasound transducer element may be configured and adapted from emitting the vibrational energy radially inwardly toward the target vessel and tissue muscle of said vessel.

### BRIEF DESCRIPTION OF THE DRAWINGS

Additional objects and features of the present invention will become more apparent and the invention itself will be best understood from the following Detailed Description of the Exemplary Embodiments, when read with reference to the accompanying drawings.

FIG. 1 is a schematic diagram of the ultrasonic vibrational energy treatment method according to the principles of the present invention.

FIG. 2 is an ultrasound treatment catheter having an external suction mechanism and an adjustable ultrasound current generator.

FIG. 3 is a cross-sectional view of the distal section of the ultrasound catheter of FIG. 2.

FIG. 4A is a perspective view of the ultrasonic treatment method for inserting a catheter into a target blood vessel.

FIG. 4B is a perspective view of the ultrasonic treatment method for deploying a suction element toward the vessel wall.

FIG. 4C is a perspective view of the ultrasonic treatment method for pulling the suction element toward the catheter for subsequent ultrasound treatment.

### DETAILED DESCRIPTION OF THE EXEMPLARY EMBODIMENTS

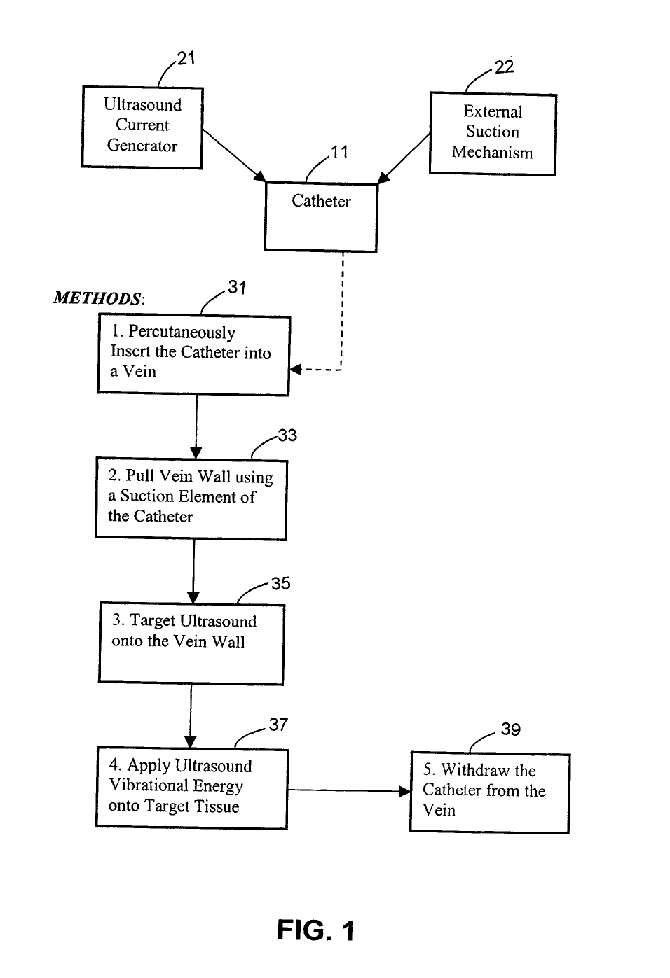

What is shown in FIG. 1 to FIG. 4C is a method for applying ultrasonic vibrational energy and a device constructed in accordance with the principles of the present invention.

FIG. 1 shows a schematic diagram of the ultrasonic vibrational energy treatment method according to the principles of the present invention. A catheter 11 of the present invention comprises at least one ultrasound transducer element and at least a suction element that is adapted for pulling the tissue toward the catheter inwardly, wherein the ultrasound transducer element is electrically coupled to an external ultrasound current generator 21 while the suction element is coupled to an external suction mechanism 22 for operations of the suction element deployment and the suction activity. In another embodiment, an expandable chamber is filled with an energy-dispersing fluid configured to enhance the energy distribution onto the tissue or muscle tissue to be treated. In an alternate embodiment, the expandable chamber enclosing the ultrasound transducer element. The method comprises dispersing the ultrasonic vibrational energy through a fluid medium of an expandable chamber. The energy-dispersing fluid of the present invention generally contains an electrolyte fluid, ion-containing fluid or the like.

The method for treating tissue of a vein, for example a varicose vein, comprises applying ultrasonic vibrational energy targeting said varicose vein effective for stimulating and restoring tissue physiological functions of the varicose vein. The method may comprise the following steps: Step #1) 31 percutaneously insert the catheter 11 into a vein 41; Step #2) 33 pull the vein wall using a suction element 17A, 17B of the catheter 11; Step #3) 35 target the ultrasound transmission onto the vein wall; Step #4) 37 apply the ultrasonic vibrational energy onto the target tissue; and Step #5) 39 withdraw the catheter 11 from the vein 41.

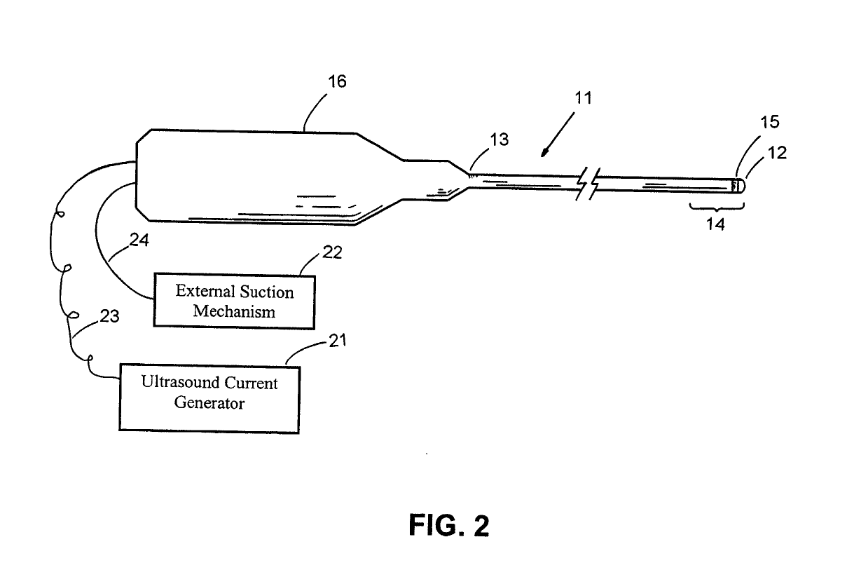

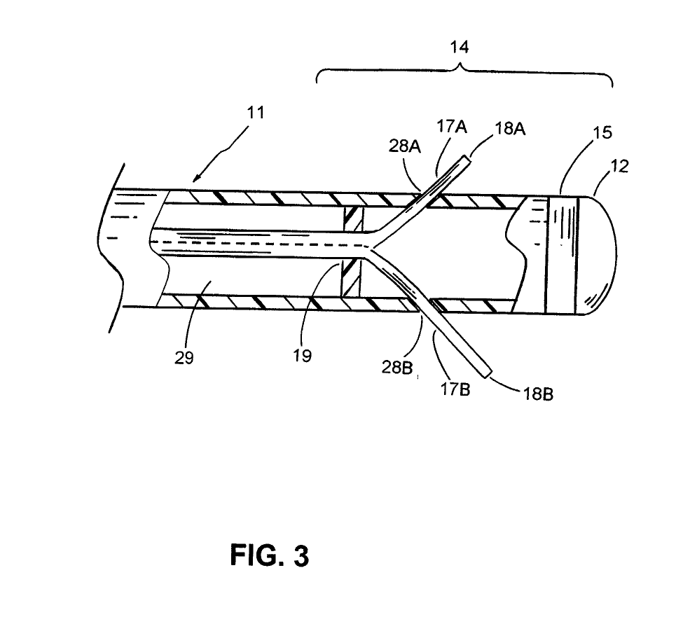

FIG. 2 shows an ultrasound treatment catheter 11 having an external suction mechanism 22 and an adjustable ultrasound current generator 21. FIG. 3 shows a cross-sectional view of the distal section 14 of the ultrasound catheter of the present invention. The catheter 11 has a catheter distal end 12, a catheter proximal end 13, a catheter distal section 14, and at least one lumen 29 extending between the catheter distal end 12 and the catheter proximal end 13, wherein there is at least one opening 28A, 28B at the catheter distal section 14 for deploying the at least one suction element 17A, 17B. A handle 16 is attached to the proximal end 3 of the catheter 11, wherein the handle has a cavity. At least one ultrasonic transducer element 15 is disposed at the distal section 14 of the catheter 11 for treating muscle tissue of a blood vessel. Alternately, the ultrasonic transducer element may be enclosed within an expandable chamber configured for enhancing the ultrasonic vibrational energy transmission. In one preferred embodiment, a portion of the expandable chamber may contact the muscle tissue of the blood vessel, for example the inner wall of a varicose vein.

The ultrasonic transducer element 15 may be a tubular transducer comprising a piezoceramic material and has an electrically conductive inner surface and an electrically conductive outer surface, a first electrical lead with a distal end portion electrically coupled to the electrically conductive inner surface and a second electrical lead with a distal end portion electrically coupled to the electrically conductive outer surface, wherein proximal end portions of the electrical leads are adapted to couple to the high frequency source.

In another preferred embodiment, the ultrasonic transducer element of the present invention may comprise an array of circumferentially spaced ultrasound transducer panels. Each ultrasound transducer panel is adapted to be individually actuated, such that each ultrasound transducer panel is configured and adapted to cause a substantial portion of the tissue stimulated.

According to one embodiment of the principles of the present invention, the ultrasound transducer element is adapted to emit a continuous circumferential pattern of ultrasonic vibrational energy whereby the tissue of the varicose vein is stimulated. In another preferred embodiment, the ultrasound transducer element is adapted to emit a discrete pattern of ultrasonic vibrational energy so as to stimulate a definite region of the target tissue or muscle tissue.

The ultrasonic vibrational energy is provided by an ultrasound transducer element connected to an external high frequency current source, wherein current of the high frequency current source is controlled by a current adjustment mechanism. In other words, the current can be controlled in an on-and-off, high-and-low, pulsed, oscilloscopic, or other appropriate manner. The frequency of the high frequency source is generally in the range of 1 MHz to 100 MHz. However, other frequency range capable of causing effective ultrasonic vibrational energy for tissue treatment is also applicable.

The external suction mechanism 22 comprises a first deployment means for deploying at least one suction element 17A, 17B out of the catheter 11 through the opening 28A, 28B. The deployment of each suction element is controlled individually. Each suction element 17A, 17B has a suction port 18A, 18B for holding the tissue by its suction force. The external suction mechanism 22 also comprises a second suction means by applying a suction on each suction port individually. A stopper 19 is generally provided at the distal section 14 of the catheter 11 to prevent fluid from leaking into the major lumen 29 of the catheter 11.

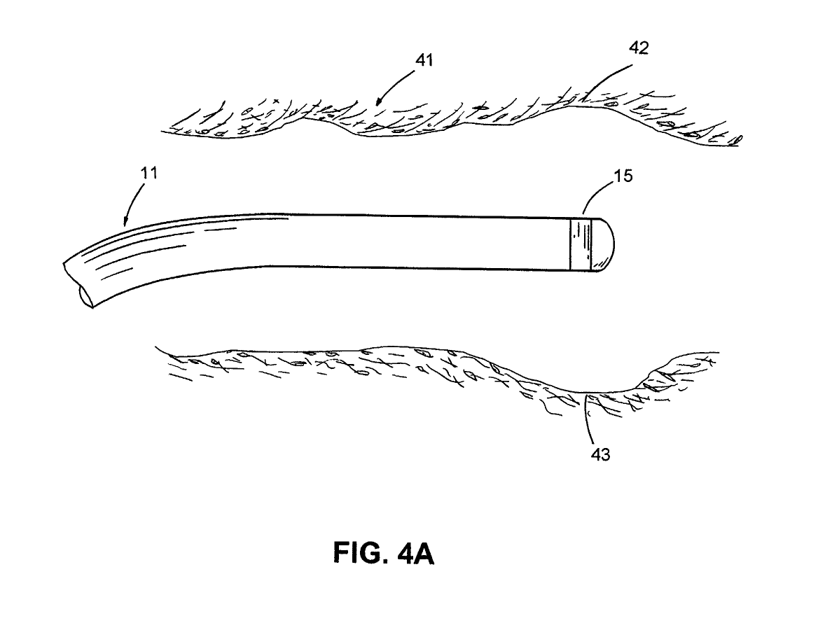

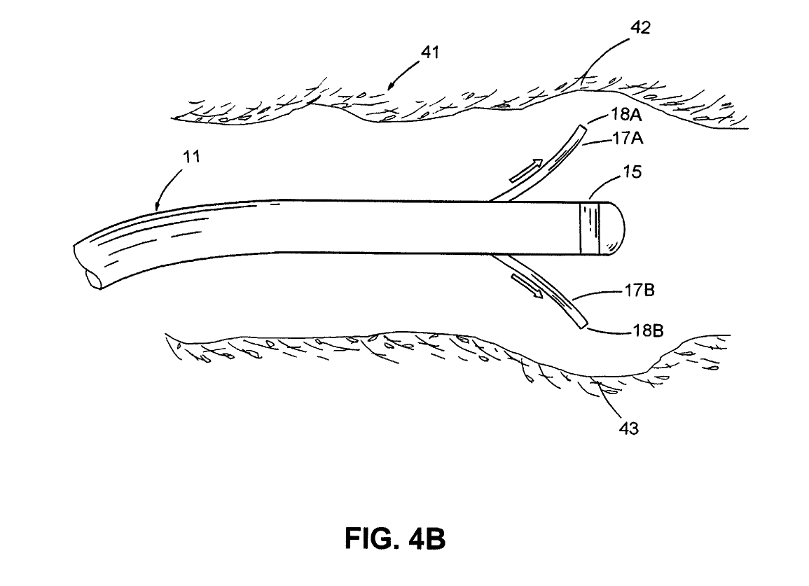

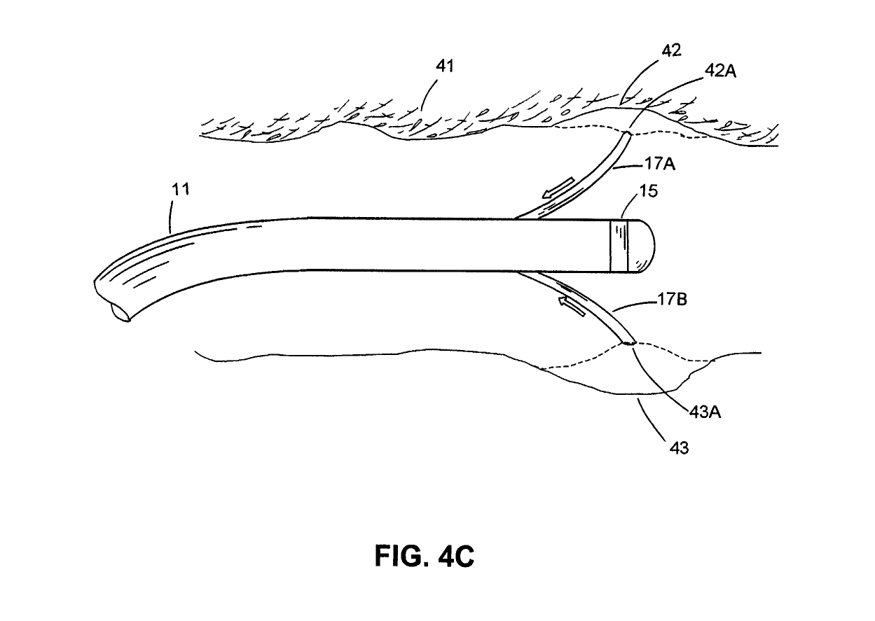

FIGS. 4A to 4C are perspective views of the ultrasonic treatment method for inserting a catheter into a blood vessel. For illustration purposes, FIG. 4A shows a first step of inserting an ultrasound catheter 11 into a blood vessel, such as a vein 41. The vein may have a varicose at 42, 43 that needs special treatment to stimulate its tissue and to restore its physiological functions. The catheter can be guided into a vessel by a guiding sheath, a guide wire or other appropriate means. FIG. 4B shows a second step that extends the suction element 17A, 17B outwardly toward the varicose 42, 43, respectively or any desired target tissue. The relative distance of the suction port 18A, 18B and the ultrasound transducer element 15 can be precalibrated. FIG. 4C shows the suction element 17A, 17B already contacts and holds the varicose tissue 42, 43 and pull the target tissue toward the catheter inwardly at a new location 42A, 43A, respectively.

For example, after the tissue 42 is pulled to a desired location, such as 42A, the ultrasonic vibrational energy may be effectively emitted from the ultrasound transducer element 15 to the tissue at a pre-determined depth and range. After treating a target tissue, the same operation method can be repeated to treat other target tissue that needs tissue stimulation.

The external ultrasonic energy generator means has the capability to supply ultrasonic energy by controlling the time, power, and temperature through an optionally separate closed-loop temperature control means. In a particular case of body temperature operation, the transducer element may be operated by a pulsed mode so that the element emits substantial vibrational energy with little heat. One material used to make piezoelectric transducers is barium titanate. The barium titanate has high sensitivity as an ultrasonic transducer, which means that it requires small voltage amplification. Barium titanate is also substantially more durable under mechanical and environmental abuse than other piezoelectric crystals and can sustain its property at a relatively high temperature. Ultrasonic vibrational energy is well known to one who is skilled in the art. U.S. Pat. No. 5,295,484, No. 5,606,974, No. 6,117,101 and No. 6,164,283 all disclose basic ultrasound principles and transducer element configurations, the entire contents of which are incorporated herein by reference.

From the foregoing, it should now be apparent that a method for providing ultrasonic vibrational energy to a tissue effective for stimulating and restoring tissue physiological functions has been disclosed. While this invention has been described with reference to a specific embodiment, the description is illustrative of the invention and is not to be construed as limiting the invention. Various modifications and applications may occur to those skilled in the art without departing from the true spirit and scope of the invention as described by the appended claims.

What is claimed is:

1. A method for treating tissue of a varicose vein, the method comprising applying ultrasonic vibrational energy targeting said varicose vein effective for stimulating and restoring tissue physiological functions of said vein.

2. The method according to claim 1, wherein the ultrasonic vibrational energy is applied from within said varicose vein.

3. The method according to claim 1, wherein the ultrasonic vibrational energy is applied from around said varicose vein.

4. The method according to claim 1, wherein said varicose vein further comprises a valve.

5. The method according to claim 1, wherein the ultrasonic vibrational energy is provided by an ultrasound transducer element connected to a high frequency current source.

6. The method according to claim 5, wherein the high frequency current source is controlled by a current adjustment mechanism.

7. The method according to claim 6, wherein frequency of the high frequency current source is in the range of 1 MHz to 100 MHz.

8. The method according to claim 5, wherein said ultrasound transducer element is mounted onto and surrounds a distal end portion of a catheter that is introduced into the varicose vein percutaneously.

9. The method according to claim 8, the method further comprising dispersing the ultrasonic vibrational energy through a fluid medium of an expandable chamber, wherein the expandable chamber encloses said ultrasound transducer element.

10. The method according to claim 8, said catheter further comprising at least a suction element adapted for pulling the tissue of the varicose vein toward a central axis of the catheter.

11. The method according to claim 5, wherein the ultrasound transducer element is a tubular transducer comprising a piezoceramic material and has an electrically conductive inner surface and an electrically conductive outer surface, a first electrical lead with a distal end portion electrically coupled to the electrically conductive inner surface and a second electrical lead with a distal end portion electrically coupled to the electrically conductive outer surface, wherein proximal end portions of the electrical leads are adapted to couple to the high frequency source.

12. The method according to claim 5, wherein the ultrasound transducer element comprises an array of circumferentially spaced ultrasound transducer panels.

13. The method according to claim 12, wherein each ultrasound transducer panel is adapted to be individually actuated, such that each ultrasound transducer panel is adapted to cause a substantial portion of the tissue stimulated.

14. The method according to claim 5, wherein the ultrasound transducer element is configured to emit a continuous circumferential pattern of ultrasonic vibrational energy whereby the tissue of the varicose vein is stimulated.

15. A catheter for treating muscle tissue of a blood vessel comprising at least one ultrasound transducer element and at least a suction element adapted for pulling the tissue toward the catheter radially.

16. The catheter according to claim 15, wherein the ultrasonic vibrational energy is provided by an ultrasound transducer element connected to a high frequency current source.

17. The catheter according to claim 16, wherein current of the high frequency current source is controlled by a current adjustment mechanism.

18. The catheter according to claim 17, wherein frequency of the high frequency source is in the range of 1 MHz to 100 MHz.

19. A method for therapeutically treating a tissue, the method comprising applying ultrasonic vibrational energy toward said tissue effective for transmitting said vibrational energy onto said tissue, wherein the vibrational energy is controlled by an adjustable high frequency generator.

20. The method according to claim 19, wherein said tissue is a pulmonary vein.
